(19) United States
(12) Patent Application Publication
(10) Pub. No.: US 2023/0384321 A1
(43) Pub. Date: Nov. 30, 2023

(54) Application of microbiological markers in non-invasive identification/early warning of fatty liver in perinatal cows

(71) Applicant: Shandong Agricultural University, Taian (CN)

(72) Inventors: Kerong SHI, Taian (CN); Haoming SUN, Taian (CN); Tingjun LIU, Taian (CN); Xuan ZHANG, Taian (CN); Xianpeng HOU, Taian (CN); Xuyang SONG, Taian (CN)

(21) Appl. No.: 18/112,518

(22) Filed: Feb. 22, 2023

(30) Foreign Application Priority Data
May 27, 2022 (CN) 202210584147.4
May 27, 2022 (CN) 202210584152.5

(51) Int. Cl. G01N 33/68

(52) U.S. Cl. CPC G01N 33/6893; G01N 33/6848; G01N 2333/195; G01N 2800/085

(57) ABSTRACT

The present disclosure discloses the application of fecal microbiological markers in non-invasive identification/early warning of fatty liver in perinatal cows, belonging to the technical field of microorganisms and clinical medicine. The microbiological marker is a combination of one or more of Bifidobacteriumpseudolongum, Prevotellamultisaccharivorax and Lachnospiraceae bacterium.

### CROSS-REFERENCES TO RELATED APPLICATION

The present application claims the benefit of the priority of the Chinese patent application with the application No. 202210584147.4, filed to the China National Intellectual Property Administration on May 27, 2022, and the Chinese patent application with the application No.202210584152.5, filed to the China National Intellectual Property Administration on May 27, 2022, the entire content of which is incorporated in this application by reference.

### TECHNICAL FIELD

The disclosure relates to the technical field of microorganisms and clinical medicine, in particular to application of microbiological markers in non-invasive identification/early warning of fatty liver in perinatal cows.

### BACKGROUND

Fatty liver is a metabolic disorder with high incidence in perinatal cows, especially in high productive cows. The period from 21 days antepartum to 21 days postpartum is called as the perinatal period (Drackley, 1999; Grummer, 1995). Perinatal cows have reduced energy intake and significantly reduced exercise amount due to the reduction of food intake, while production and galactosis require a lot of energy support, which will lead to fat mobilization, resulting in the body consuming its own fat, the concentration of non-esterified fatty acids (NEFAs) in the blood is increased, and the rate of non-esterified fatty acids uptake by the liver is accelerated. These NEFAs are esterified into triglycerides (TGs) after entering the liver, TG can be secreted out of the liver by hydrolysis or in the form of very low density lipoproteins (VLDLs); however, the efficiency of VLDLs secreted into blood is very low. When the synthesis speed of TG is higher than the transportation speed, it will lead to the accumulation of TG in the liver, so that the accumulation of fat in the liver exceeds the normal content in the liver, which is called fatty liver. Fatty liver disease of cows is one of the metabolic disorders that often occur in the perinatal period of cows. In serious cases, it can also cause ketosis, postpartum paralysis, etc., which seriously affects the milk production performance, reproductive performance and service life in cows. The incidence of this disease is particularly common in the perinatal period of cows, and the incidence rate is relatively high (5-10% of cows suffer from severe fatty liver, 30-40% suffer from moderate or mild fatty liver), causing huge economic losses to the dairy industry. Therefore, accurate diagnosis of fatty liver in perinatal cows is not only beneficial to the health of cows, but also can reduce the economic loss of pastures.

At present, the detection methods of fatty liver mainly include liver biopsy, serum physiology and biochemistry, proteomics and metabonomics, digital image technology, etc. Among them, the liver biopsy, i.e. the determination of fat content in liver tissue of living cow is an important inspection method for liver metabolism research. At present, the gold standard for the diagnosis of fatty liver in cows is determined by measuring the proportion of triglycerides in the liver to the wet weight of the liver. TG<1% in liver of cows is normal; 1%<TG<5% is mild fatty liver; 5%<TG<10% is moderate fatty liver; and TG>10% is severe fatty liver. However, this method is an invasive method, which has an even worse impact on the health of cows and is not conducive to animal welfare. Moreover, poor prognosis will also lead to complicated infectious diseases. In clinical practice, fatty liver in cows can also be diagnosed through serum physiology and biochemistry. At present, the well approved diagnostic method is the Y value method proposed by Reid (calculated by three serum biochemical indicators), but there is still a large error after investigation, which is not suitable for large-scale pastures.

In the previous study, the inventor found that the small molecule metabolite in feces was used as a marker (L-α-aminobutyric acid and behenic acid in feces disclosed in the patent with Application No. 201910873316.4, and 3-nitrotyrosine in urine as biomarkers, and can be used for non-invasive diagnosis of fatty liver in cows. However, the determination process of small molecule metabolites is relatively complex, and the abundance and stability of small molecule metabolites are relatively low. At the same time, both cow feces and cow urine are needed, but the collection of urine is more troublesome. Therefore, the search for new fecal markers for non-invasive identification or early warning of fatty liver in cows is still the bottleneck urgently needed to be broken through in the prevention and treatment of fatty liver diseases in perinatal cows.

### SUMMARY

The object of the disclosure is to provide a group of microbiological markers and the application in non-invasive identification/early warning of fatty liver in perinatal cows thereof. Proven by diagnostic capability, the AUC values of the microbiological markers of the disclosure conform to the diagnostic significance and has high clinical diagnostic application value; the microbiological marker of the disclosure can be used to diagnose, identify and monitor the perinatal cows with fatty liver. It is not only of low cost, simple operation, but also a non-invasive and non-aggressive detection method, which conforms to the concept of animal welfare and healthy breeding, and can be widely used in the large-scale breeding of cows.

In order to achieve the above object, the disclosure adopts the following technical solution:

In a first aspect of the disclosure, use of a microorganism of at least one species of the following 1)-3) as a microbiological marker for preparation of a reagent or a kit for diagnosing the fatty liver diseases in perinatal cows is provided:
1) Bifidobacteriumpseudolongum;
2) Prevotellamultisaccharivorax;
3) Lachnospiraceae bacterium.

The above three microbiological markers are isolated from cow feces, which can accurately identify fatty liver diseases in cows. The AUC values of each kind of microbiological markers are consistent with diagnostic significance, and have high clinical diagnostic application value. Moreover, AUC is closer to 1 and has better diagnostic effect than single AUC when multiple AUCs are used together. When the above three microbiological markers are used in combination, the recognition effect of fatty liver diseases in perinatal cows is the best.

Therefore, preferably, the microbiological marker is a combination of the three Bifidobacteriumpseudolongum, Prevotellamultisaccharivorax and Lachnospiraceae bacterium.
or, the microbiological marker is a combination of the two Bifidobacteriumpseudolongum and Lachnospiraceae bacterium.

In a second aspect of the disclosure, use of a reagent for detecting microbiological markers in cows' metabolites in the preparation of products for non-invasively identifying fatty liver diseases in perinatal cows is provided;
the microbiological marker is a combination of one or more of Bifidobacteriumpseudolongum, Prevotellamultisaccharivorax and Lachnospiraceae bacterium.

Preferably, the microbiological marker is a combination of the three Bifidobacteriumpseudolongum, Prevotellamultisaccharivorax and Lachnospiraceae bacterium;
or, the microbiological marker is a combination of the two Bifidobacteriumpseudolongum and Lachnospiraceae bacterium.

Further, the reagent is one for detecting the relative abundance of the microbiological markers in cows' metabolites.

Preferably, the reagent is one for Metagenomics sequencing, 16S sequencing, qPCR sequencing or Metaproteomics sequencing.

Preferably, the cows' metabolite is feces.

In the use described above, the method for non-invasively identifying fatty liver diseases in perinatal cows comprises:
(1) collecting feces from perinatal cows to be tested;
(2) detecting the relative abundance of microbiological markers in cows' feces; and
(3) identifying whether the perinatal cows to be tested have fatty liver diseases based on the relative abundance of the detected microbiological markers.

In a third aspect of the disclosure, use of a microbiological marker in feces in the preparation of a reagent or a kit for diagnosing the fatty liver diseases in perinatal cows is provided, the microbiological marker is selected from a combination of at least two of Lachnoanaerobaculum, Roseburia, and Bifidobacterium.

Preferably, the microbiological marker is a combination of Lachnoanaerobaculum, Roseburia, and Bifidobacterium.

In a fourth aspect of the disclosure, use of a reagent for detecting microbiological markers in cows' metabolite feces in the preparation of products for non-invasively identifying fatty liver diseases in perinatal cows is provided;
the microbiological marker is selected from a combination of at least two of Lachnoanaerobaculum, Roseburia, and Bifidobacterium.

Preferably, the microbiological marker is a combination of Lachnoanaerobaculum, Roseburia, and Bifidobacterium.

Preferably, the reagent is one for detecting the relative abundance of the microbiological markers in cows' feces.

Preferably, the reagent is one for Metagenomics sequencing, 16S sequencing, qPCR sequencing or Metaproteomics sequencing.

Preferably, the method for non-invasively identifying fatty liver diseases in perinatal cows comprises:
(1) collecting feces from perinatal cows to be tested;
(2) detecting the relative abundance of microbiological markers in cows' feces; and
(3) identifying the fatty liver diseases of the perinatal cows to be tested based on the relative abundance of the detected microbiological markers for diagnosis or early warning.

The beneficial effects of the disclosure:
(1) The disclosure is based on macroproteomics technology, and for the first time proposes a microbiological marker for non-invasive recognition and identification of cows with fatty liver disease at the species level. Through the verification of diagnostic ability, the AUC of each of microbiological markers of the disclosure is higher, and has higher clinical diagnostic application value.
(2) The microbiological marker of the disclosure is used to recognize, identify and monitor the cows with fatty liver. It is not only of low cost, simple operation, but also a non-invasive and non-aggressive detection method, which conforms to the concept of animal welfare and healthy breeding, and can be widely used in the large-scale breeding of cows in the future, promoting the healthy and efficient development of dairy industry.
(3) The disclosure uses fecal microbiological markers, and feces are the end metabolites of cows, which not only can reflect the metabolic status of the body, but also can “non-invasively” diagnose/early warning the metabolic status of cows. The secondary injury to cows caused by liver biopsy is avoided; and the cumbersome steps and inconvenience caused by binding cows during serum collection is also avoided. In addition, compared with the fecal small molecule metabolites discovered by the inventor in the early stage, the abundance of the fecal microbiological markers are higher and more stable than that of the small molecule metabolites, which is conducive to ensuring the specificity and sensitivity of the markers. Moreover, the determination of the fecal microorganisms is simpler than that of small molecule metabolites.

### BRIEF DESCRIPTION OF THE DRAWINGS

The accompanying drawings, which form a part of this application, are provided to further understand the present disclosure, the illustrative embodiments of the present disclosure and the description thereof are intended to explain the present disclosure and are not intended to limit thereto. In the drawings:

FIG. 1: Random Forest analysis of fecal microorganism Bifidobacteriumpseudolongum in MFLvsNorm.

FIG. 2: Random Forest analysis of fecal microorganism Bifidobacterium pseudolongum in SFLvsNorm.

FIG. 3: In MFLvsNorm group, different fecal microorganisms and their combinations are used for combined analysis in ROC; Among them, B represents Bifidobacteriumpseudolongum, L represents Lachnospiraceae bacterium, P represents Prevotellamultisaccharivorax; B+L+P represents combined use of Bifidobacteriumpseudolongum, Lachnospiraceae bacterium and Prevotellamultisaccharivorax; B+L represents combined use of Bifidobacteriumpseudolongumand Lachnospiraceae bacterium; B+P represents combined use of Bifidobacteriumpseudolongum and Prevotellamultisaccharivorax; P+L represents combined use of PrevotellamultisaccharivoraxandLachnospiraceae bacterium.

FIG. 4: In SFLvsNorm group, different fecal microorganisms and their combinations are used for combined analysis in ROC; Among them, B represents Bifidobacteriumpseudolongum, L represents Lachnospiraceae bacterium, P represents Prevotellamultisaccharivorax; B+L+P represents combined use of Bifidobacteriumpseudolongum, Lachnospiraceae bacterium and Prevotellamultisaccharivorax; B+L represents combined use of Bifidobacteriumpseudolongumand Lachnospiraceae bacterium; B+P represents combined use of Bifidobacteriumpseudolongum and Prevotellamultisaccharivorax; P+L represents combined use of PrevotellamultisaccharivoraxandLachnospiraceae bacterium.

FIG. 5. In MFLvsNorm group, Microorganism Roseburia+Bifidobacterium is used for combined analysis in ROC by MFLvsNorm.

FIG. 6. In SFLvsNorm group, Microorganism Lachnoanaerobaculum+Bifidobacterium is used for combined analysis in ROC.

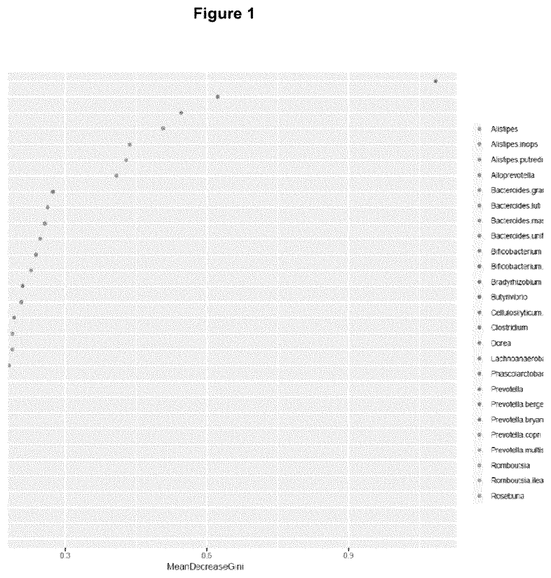

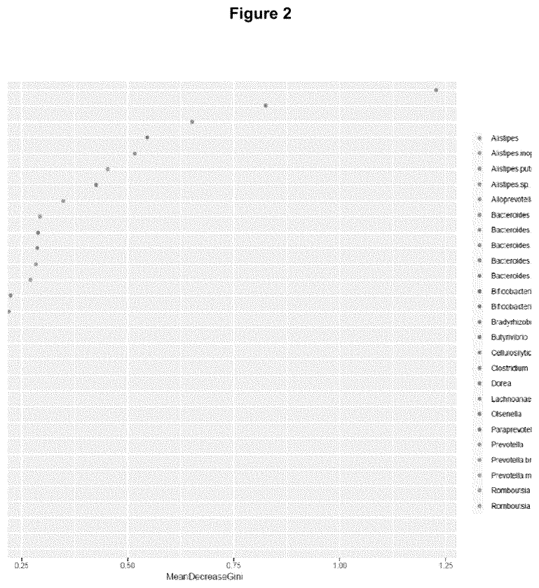

### DETAILED DESCRIPTION OF THE EMBODIMENTS

It should be noted that the Examples in the present application and the features in the Examples may be combined with each other without conflicting. Hereinafter, the present disclosure will be described in detail with reference to the drawings and in conjunction with the Examples.

As described above, fatty liver disease of cows is one of the metabolic disorders that often occur in the perinatal period of cows. In serious cases, it can also cause ketosis, postpartum paralysis, etc., which seriously affects the milk production performance, reproductive performance and service life in cows. Huge economic losses are caused to the dairy industry. If we can identify in advance or have corresponding early warning measures, such losses can be effectively avoided. At present, the only reliable diagnostic method is liver biopsy, in which liver tissue of living cows is taken for fat content determination. However, this method is an invasive method, which has an even worse impact on the health of cows and is not conducive to animal welfare. Moreover, poor prognosis will also lead to complicated infectious diseases. Therefore, it is of great significance and value to use non-invasive methods for early diagnosis of fatty liver in cows.

Proteome covers a wide range, including food, ocean, soil and intestinal tract, etc. With the improvement and development of sample processing and mass spectrometry in recent years, the combination of macroproteomics and bioinformatics is becoming more and more mature.

Macroproteomics also showed great changes in the analysis of intestinal changes caused by NAFLD. In particular, it can reveal changes in the classification, function and metabolic pathway of intestinal microbiota.
(1) The disclosure adopts 4D proteomics technology to conduct macroproteomics analysis on feces. The advantage of macroproteomics lies in the comprehensive analysis of the classification and function of microorganisms in feces.

Specific steps are as follows:
\textcircled{1} Protein extraction: The sample is taken out at −80° C., a proper amount of tissue sample is weighed into a mortar pre-cooled by liquid nitrogen, which is ground with liquid nitrogen to powder. Phenol extraction buffer (containing 10 mMdithiothreitol, 1% protease inhibitor,) that is 4 times the volume of the powder is added to the samples in each group, respectively, then subjected to ultrasonic lysis. An equal volume of Tris balance phenol is added, centrifuged at 4° C. for 10 min at 5500 g, the supernatant is taken and 0.1 M ammonium acetate/methanol that is 5 times the volume of the supernatant is added to precipitate overnight, the protein precipitate is washed with methanol and acetone, respectively. Finally, the precipitate is redissolved with 8 M urea, and the protein concentration is determined with BCA kit.

\textcircled{2} Pancreatinenzymolysis: The same amount of protein is taken from each sample for enzymolysis, an appropriate amount of standard protein is added, the volume is adjusted to be consistent by the lysate, TCA with a final concentration of 20% is slowly added, vortex mixed well, and precipitatied at 4° C. for 2 h. Then it is centrifuged for 5 min at 4500 g, the supernatant is discarded, and the precipitate is washed with the pre-cooled acetone for 2-3 times. After the precipitate is air-dried, TEAB with a final concentration of 200 mM is added, the precipitate is dispersed by ultrasonic, trypsin is added in a proportion of 1:50 (protease:protein, m/m), and subjected to enzymolysis overnight. Dithiothreitol (DTT) is added to make its final concentration 5 mM, which is then reduced for 30 min at 56° C. After which the iodoacetamide (IAA) is added to make its final concentration 11 mM.
\textcircled{3} Analysis by liquid chromatography-mass spectrometry. The peptide segment is dissolved in liquid chromatography mobile phase A (0.1% (v/v) aqueous formic acid solution), and then separated by EASY-nLC 1000 ultra high performance liquid phase system. Mobile phase A is an aqueous solution containing 0.1% formic acid and 2% acetonitrile; Mobile phase B is an aqueous solution containing 0.1% formic acid and 90% acetonitrile. Liquid phase gradient setup: 0-40 min, 5%-25% B; 40-52 min, 25%-35% B; 52-56 min, 35%-80% B; 56-60 min, 80% B, flow rate maintained at 500 nL/min.

The peptide segment is separated by an ultra high performance liquid phase system, and then injected into an NSI ion source for ionization, and then analyzed by Q Exactive™ Plus mass spectrometry. The ion source voltage is set to be 2.1 kV, and the peptide segment parent ions and its secondary fragments are detected and analyzed using high-resolution Orbitrap. The scanning range of the primary mass spectrometer is set to be 350-1800 m/z, and the scanning resolution is set to be 70,000; the fixed starting point of the scanning range of the secondary mass spectrometry is 100 m/z, and the secondary scanning resolution is set to be 17,500. The data acquisition mode uses the Data Dependent Acquisition (DDA) program, that is, after the primary scanning, the parent ions of the first 10 peptide segments with the highest signal strength are selected to enter the HCD collision pool in turn, and 28% of the fragmentation energy is used for fragmentation, and the secondary mass spectrometry analysis is also performed in turn. In order to improve the effective utilization of mass spectrometry, the automatic gain control (AGC) is set to be 5E4, the signal threshold is set to be 20000 ions/s, the maximum implantation time is set to be 100 ms, and the dynamic exclusion time of tandem mass spectrometry scanning is set to be 30 s to avoid repeated scanning of parent ions.
\textcircled{4} Database search and comparison: The secondary mass spectrometry data are retrieved using Maxquant (v1.5.2.8). Retrieval parameter setting: the database has added an reverse database to calculate the false positive rate (FDR) caused by random matching, and the database has added a common pollution database to eliminate the impact of contaminated proteins in the identification results; the digestion method is set to Trypsin/P; the number of missed digestion sites is set to 2; the tolerance of the quality error of the primary parent ions of First search and Main search is set to 20 ppm and 5 ppm, respectively, and the tolerance of the quality error of the secondary fragment ions is 0.02 Da. The alkylation of cysteine is set as fixed modification, and the variable modification is the oxidation of methionine, acetylation and deamidization (NQ) at N-terminal of proteins. The FDR of protein identification and PSM identification is set to 1%.
\textcircled{2} Firstly, the metaproteomics data shall be subject to data repeatability test and mass spectrometry quality control test firstly, and the unqualified or unreasonable data shall be removed; Pearson correlation coefficient and OPLS-DA method are used to verify the reliability of the screening model, and then the differentially expressed proteins between normal and diseased groups are screened. Software Unipept (v.2.0.0 https:l/unipept.ugent.be/datasets) and Mass spectrometry data analysis MaxQuant (v.1.5.2.8 http://www.maxquant.org/) are used for species annotation.
(3) The biomarker in the disclosure has undergone a rigorous screening process.
(4) 31 cow individuals are analyzed with macroproteomics. Compared with other methods of identifying fatty liver individuals by fecal microbiological indicators, the microorganisms screened at species level have good verification ability.

The disclosure finally finds three microbiological markers at species level with diagnostic values: Bifidobacteriumpseudolongum, Prevotellamultisaccharivorax and Lachnospiraceae bacterium.

The three microbiological markers of the disclosure have high discrimination ability in identifying NormvsMFL. In addition, it is found that Bifidobacteriumpseudolongum in feces has a high discrimination ability in both NormvsMFL and NormvsSFL groups. Bifidobacteriumpseudolongum might have a positive correlation with the thickness of intestinal mucosa. Because the mucus layer plays a crucial role in intestinal tract protection, it is important to reveal the importance of studying mucus degrading bacteria for understanding the potential causes of diseases such as intestinal diseases, etc., and implementing new treatment strategies.

The disclosure also finds three microbiological markers at species level with diagnostic values in another group of 31 cow individuals: Lachnoanaerobaculum, Roseburia, and Bifidobacterium.

The six microbiological markers with diagnostic values found in the disclosure derive from feces, which can be identified by microorganisms generated by the normal metabolism of cows, it is of time saving, convenience and diagnostic cost saving. It does not require traditional blood sample collection or surgical puncture when diagnosing, which is a painless identification marker and is of great significance to the health and safety of cows and animal welfare. Using the marker of the disclosure to identify and diagnose fatty liver diseases in cows will not affect milk production of cows, and will not produce negative effects such as declined dairy production, health stress or even accompanying infections caused by blood sample collection, and surgical puncture, etc. The diagnosis and treatment costs are saved, and high yield and efficiency are promoted.

The biomarker used in the disclosure derives from the feces and is a microorganism in the intestinal tract of cows, which can well indicate the metabolism of cows. For the fatty liver of cows, it can accurately indicate the metabolism of cows through the change of the relative abundance of the microorganism content in the feces.

In order to enable those skilled in the art to understand the technical solution of the application more clearly, the technical solution of the application will be described in detail in combination with specific examples.

The test materials used in the examples of the disclosure that have not been specifically described are conventional test materials in the art and commercially available.

### Example 1: Screening and Discovery of the First Group of Candidate Markers (Screening and Discovery of Metabolite Differential Markers of Liver Biopsy Diagnostic Population)

First, 31 cows were selected as the Discovery set. Through liver biopsy, they were divided into normal group (Norm, n=10) and moderate fatty liver group (MFL, n=9) and severe fatty liver group (SFL, n=12).

Feces of cow were collected, the corresponding signal abundance of protein in each sample was detected by mass spectrometry, and the LFQ intensity of protein in each sample was obtained by non-standard quantitative calculation method, and the relative quantitative value of each sample was obtained according to the LFQ intensity of proteins between different samples.

The first step was to calculate the differential expression amount of proteins between two samples in the comparison group. First, the average values of quantitative values of each sample in multiple repetitions were calculated, and then the ratio of the average value between two samples was calculated. This ratio was used as the final differential expression amount of the comparison group.

The second step was to calculate the differential expression significance P-value of the protein in the two samples. First, the relative quantitative value of each sample was undergone log 2 (so that the data conformed to the normal distribution), and then p-value was calculated using the two sample two tailed T-test method. When p-value was less than 0.05, the changed threshold value for significant up-regulation and the changed threshold value for significant down-regulation were those with differential expression changes exceeding 1.5 and less than 1/1.5, respectively.

Table 1 shows the summary data of all differentially expressed proteins in this project.

TABLE 1
Statistics of differentially expressed proteins

| Comparative | Amount of up-regulated | Amount of down-regulated |
|---|---|---|
| groups | proteins (fold change > 1.5) | proteins (fold change < 1/1.5) |
| MFL/Norm | 117 | 101 |
| SFL/MFL | 170 | 277 |
| SFL/Norm | 183 | 177 |

### Example 2: In the Normal Group (Norm) and the Diseased Group, Almost all the Proteins with the Largest Fold Change Derived from the Microorganism Bifidobacteriumpseudolongum

In MFLvsNorm, 218 differentially expressed protein proteins were screened from the metaproteomics, and ten proteins with the largest up-regulated fold change were screened, among which A0A0A7ICF4 with the largest fold change reached 70.48, and all of these proteins were derived from Bifidobacteriumpseudolongum (as shown in Table 2). Similarly, in the SFLvsNorm group, we observed that F3BBL3, the protein with the largest fold change among the top 10 proteins in terms of the fold change was derived from Lachnospiraceae bacterium with a fold change of 48.37. The other protein was derived from Treponemaparaluiscuniculi, and the remaining proteins were derived from Bifidobacteriumpseudolongum (as shown in Table 3).

TABLE 2
Top 10 proteins in terms of fold change in MFLvsNorm group

| | | MFL/ | MFL/ |
|---|---|---|---|
| Protein | | Norm | Norm |
| Accession No. | microorganism | Ratio | P value |
| A0A0A7ICF4 | Bifidobacterium pseudolongum | 70.4869 | 0.028 |
| A0A4Q5A9Z9 | Bifidobacterium pseudolongum | 67.9707 | 0.040 |
| A0A2N3QYU5 | Bifidobacterium pseudolongum | 51.3377 | 0.007 |
| A0A0A7IBV2 | Bifidobacterium pseudolongum | 47.664 | 0.040 |
| Q6R2Q6 | Bifidobacterium pseudolongum | 29.0344 | 0.017 |
| A0A2N3QW86 | Bifidobacterium pseudolongum | 27.9436 | 0.042 |
| A0A2N3QV65 | Bifidobacterium pseudolongum | 20.562 | 0.049 |
| A0A2N3QYR2 | Bifidobacterium pseudolongum | 18.9801 | 0.048 |
| A0A2N3QY09 | Bifidobacterium pseudolongum | 17.2143 | 0.010 |
| A0A4Q5B7W4 | Bifidobacterium pseudolongum | 14.7774 | 0.037 |

TABLE 3
Top 10 proteins in terms of fold change in SFLvsNorm group

| Protein | | SFL/Norm | SFL/Norm |
|---|---|---|---|
| Accession No. | microorganism | Ratio | P value |
| F3BBL3 | Lachnospiraceae bacterium | 48.37 | 0.018 |
| A0A4Q5A9Z9 | Bifidobacterium pseudolongum | 41.815 | 0.020 |
| A0A0A7ICF4 | Bifidobacterium pseudolongum | 25.4028 | 0.011 |
| A0A2N3QW86 | Bifidobacterium pseudolongum | 20.352 | 0.027 |
| Q6R2Q6 | Bifidobacterium pseudolongum | 18.8455 | 0.045 |
| A0A4Q5B8Z0 | Bifidobacterium pseudolongum | 17.693 | 0.048 |
| A0A2N3QYU5 | Bifidobacterium pseudolongum | 16.6443 | 0.032 |
| F7XRB4 | Treponema paraluiscuniculi | 14.6661 | 0.043 |
| A0A2N3QYR2 | Bifidobacterium pseudolongum | 13.5092 | 0.031 |
| A0A2N3QST7 | Bifidobacterium pseudolongum | 11.9288 | 0.044 |

### Example 3: Verification of Microorganism Identification Ability by ROC Curve and Random Forest Analysis

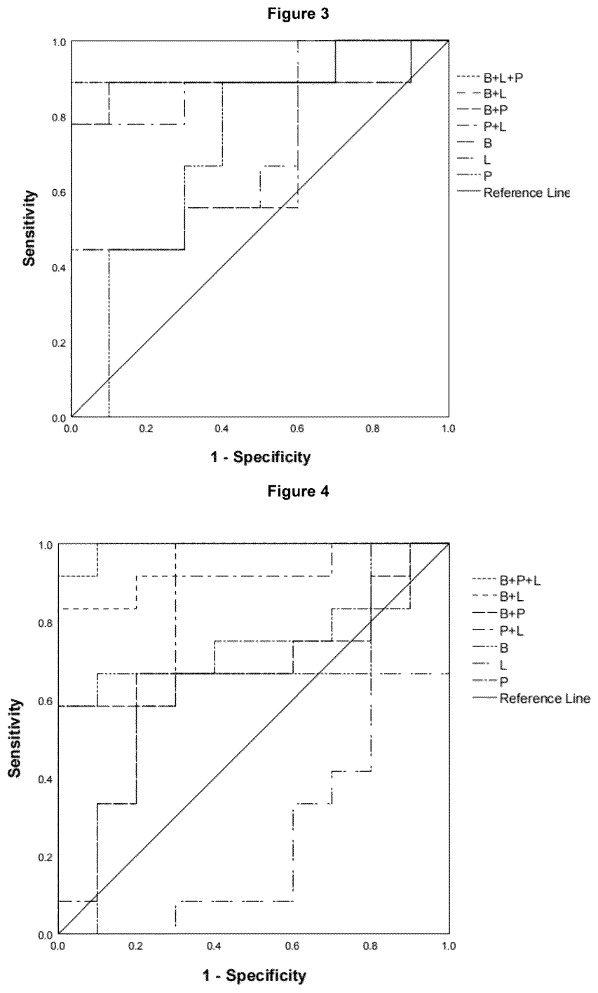

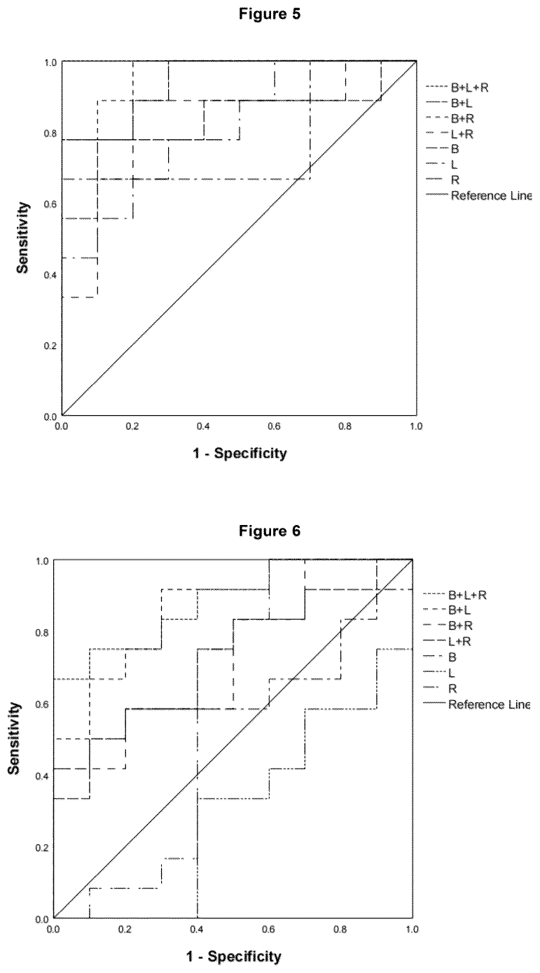

First, the top 40 microorganisms with the abundance at the species level were screened for random forest analysis. In FIG. 3, we observed that Bifidobacterium pseudolongum had the highest score on the X-axis, which also proved that Bifidobacteriumpseudolongum had a high distinguishing ability between MFL group and Norm group. Similarly, we also observed that Bifidobacterium pseudolongum ranked higher, at fourth in FIG. 4.

The ROC analysis in SPSS was used to analyze the abundance changes in microorganisms, and Bifidobacteriumpseudolongum, Prevotellamultisaccharivorax and Lachnospiraceae bacterium in feces at species level were found. Compared with other microorganisms, it has higher validation ability, as shown in Table 4. Similarly, Bifidobacteriumpseudolongum also had good results in SFLvsNorm, but AUC values of the other two microorganisms were not ideal (Table 5).

TABLE 4
Validation of Accuracy of Fecal Microorganisms at Species Level
In Diagnosing the Metabolic Status of Cows (MFLvsNorm)¹

| | | AUC P | MFL/Norm P |
|---|---|---|---|
| microorganism | AUC | value | value |
| Bifidobacteriumpseudolongum | 0.867 | 0.007 | 0.078 |
| Prevotellamultisaccharivorax | 0.722 | 0.102 | 0.360 |
| Lachnospiraceae bacterium | 0.711 | 0.121 | 0.046 |
| Turicibactersanguinis | 0.689 | 0.165 | 0.250 |
| Bifidobacteriumthermacidophilumsubspthermacidophilum | 0.678 | 0.191 | 0.149 |
| Prevotellacopri | 0.667 | 0.221 | 0.182 |
| Ruminococcaceae bacterium | 0.622 | 0.369 | 0.248 |
| Romboutsiailealis | 0.622 | 0.369 | 0.222 |
| Clostridiumhiranonis | 0.6 | 0.462 | 0.297 |
| Treponemabryantii | 0.578 | 0.568 | 0.404 |
| Rikenellaceaebacterium | 0.556 | 0.683 | 0.250 |
| Treponemaporcinum | 0.5 | 1 | 0.404 |
| Prevotellabergensis | 0.478 | 0.87 | 0.206 |
| Prevotellaruminicola | 0.456 | 0.744 | 0.185 |
| Cellulosilyticumlentocellum | 0.456 | 0.744 | 0.239 |
| Bacteroidesmassiliensis | 0.4 | 0.462 | 0.173 |
| Bacteroidesuniformis | 0.333 | 0.221 | 0.299 |
| Prevotellabryantii | 0.333 | 0.221 | 0.353 |
| Bacteroidesgraminisolvens | 0.233 | 0.05 | 0.027 |
| Prevotellaceae bacterium | 0.2 | 0.027 | 0.021 |

¹Note:
Top 20 Microorganisms with Abundance at Species Level of Fecal Microorganisms in Cows

TABLE 5
Validation of Accuracy of Fecal Microorganisms at Species Level
In Diagnosing the Metabolic Status of Cows (SFLvsNorm)¹

| | | AUC P | SFL/Norm P |
|---|---|---|---|
| taxonomy | AUC | value | value |
| Bacteroidesgraminisolvens | 0.817 | 0.012 | 0.012 |
| Bifidobacteriumpseudolongum | 0.725 | 0.075 | 0.055 |
| Treponemaporcinum | 0.692 | 0.129 | 0.082 |
| Treponemabryantii | 0.675 | 0.166 | 0.072 |
| Prevotellamultisaccharivorax | 0.65 | 0.235 | 0.495 |
| Bacteroidesuniformis | 0.633 | 0.291 | 0.210 |
| Romboutsiailealis | 0.592 | 0.468 | 0.433 |
| Prevotellacopri | 0.583 | 0.51 | 0.447 |
| Prevotellabergensis | 0.483 | 0.895 | 0.140 |
| Clostridiumhiranonis | 0.475 | 0.843 | 0.491 |
| Turicibactersanguinis | 0.458 | 0.742 | 0.206 |
| Prevotellaruminicola | 0.458 | 0.742 | 0.205 |
| Prevotellaceae bacterium | 0.4 | 0.429 | 0.485 |
| Rikenellaceae bacterium | 0.367 | 0.291 | 0.182 |
| Ruminococcaceae bacterium | 0.333 | 0.187 | 0.084 |
| Bacteroidesmassiliensis | 0.308 | 0.129 | 0.164 |
| Bifidobacteriumthermacidophilum | 0.3 | 0.114 | 0.063 |
| Prevotellabryantii | 0.233 | 0.035 | 0.043 |
| Lachnospiraceae bacterium | 0.233 | 0.035 | 0.011 |
| Cellulosilyticumlentocellum | 0.267 | 0.065 | 0.062 |

¹Note:
Top 20 Microorganisms with Abundance at Species Level of Fecal Microorganisms in Cows

The ROC in SPSS was used to analyze, and the microorganism Bifidobacteriumpseudolongum in MFLvs Norm and SFLvsNorm groups was found in microorganisms at species level. The area under the curve (AUC) in both groups was greater than 0.7, and it was as high as 0.867 in MFLvsNorm (Table 4), with high validation ability. In SFLvsNorm, the area under the curve also reached 0.725 (Table 5).

### Example 4: Abundance Change of Microorganisms in Three Groups of Norm-MFL-SFL

In the ROC curve, the abundance changes of Bifidobacteriumpseudolongum, Prevotellamultisaccharivorax and Lachnospiraceae bacterium were shown in Table 6. The abundance of Lachnospiraceae bacterium in Norm is 3.55E+10, and the increase in abundance was nearly doubled in MFL. Similarly, the abundance of Bifidobacteriumpseudolongum in both MFL and SFL statuses increased nearly 10 times, especially increased more in MFL.

Among them, the abundance of Lachnospiraceae bacterium accounted for 55.78% in Norm and 66.00% in MFL. The abundance of Bifidobacteriumpseudolongum accounted for 0.73% in Norm, and increased to 6% in MFL and SFL (Table 7).

TABLE 6
Top 20 Microorganisms with Abundance at Species Level (mean ± standard deviation)

| Microorganisms | Norm | MFL | SFL |
|---|---|---|---|
| Lachnospiraceae | 3.55E+10±1.48E+10 | 6.12E+10±3.67E+10 | 2.1E+10±1.02E+10 |
| bacterium | | | |
| Ruminococcaceae | 7.25E+09±1.42E+09 | 7.68E+09±1.17E+09 | 6.38E+09±1.25E+09 |
| bacterium | | | |
| Bifidobacteriumpseudolongum | 4.63E+08±9.2E+07 | 5.84E+09±9.76E+09 | 2.82E+09±4.51E+09 |
| Prevotellabryantii | 4.51E+09±4.72E+09 | 3.54E+09±5.62E+09 | 1.64E+09±9.69E+08 |
| Prevotellaruminicola | 3.08E+09±2.06E+09 | 2.37E+09±1.01E+09 | 2.45E+09±8.89E+08 |
| Rikenellaceae | 2.06E+09±7.4E+08 | 2.24E+09±6.23E+08 | 1.79E+09±5.26E+08 |
| bacterium | | | |
| Turicibactersanguinis | 1.61E+09±9.85E+08 | 1.87E+09±4.88E+08 | 1.29E+09±6.17E+08 |
| Prevotellaceae | 1.11E+09±3.57E+08 | 8.14E+08±1.72E+08 | 1.1E+09±7.28E+08 |
| bacterium | | | |
| Romboutsiailealis | 8.46E+08±5.46E+08 | 1.02E+09±3.46E+08 | 8.82E+09±3.49E+08 |
| Prevotellamultisaccharivorax | 8.82E+08±8.62E+08 | 9.91E+08±1.93E+08 | 8.85E+08±4.24E+08 |
| Bacteroidesgraminisolvens | 6.89E+08±5.55E+08 | 2.7E+08±1.7E+08 | 1.26E+09±4.83E+08 |
| Prevotellacopri | 7.66E+08±5.13E+08 | 9.96E+08±5.03E+08 | 7.94E+08±3.9E+08 |
| Cellulosilyticumlentocellum | 1.11E+09±1.02E+09 | 8.37E+08±4.72E+08 | 5.09E+08±4.42E+08 |
| Bacteroidesuniformis | 7.11E+08±7.02E+08 | 5.11E+08±8.17E+08 | 9.6E+08±6.38E+08 |
| Bacteroidesmassiliensis | 9.01E+08±4.54E+08 | 7.25E+08±2.79E+08 | 7.14E+08±3.59E+08 |
| Treponemabryantii | 5.37E+08±2.26E+08 | 5.6E+08±1.64E+08 | 7.57E+08±4.07E+08 |
| Treponemaporcinum | 5.45E+08±4.42E+08 | 4.64E+08±2.39+08 | 9.62E+08±8.17E+08 |
| Clostridium hiranonis | 3.16E+08±2.02E+08 | 3.68E+08±1.91E+08 | 3.19E+08±2.63E+08 |
| Bifidobacteriumthermacidophilum | 2.1E+07±1.5E+07 | 3.0E+07±1.9E+07 | 1.1E+07±1.2E+07 |
| Prevotellabergensis | 7.52E+08±1.15E+09 | 3.9E+08±5.37E+08 | 3.06E+08±2.25E+08 |

TABLE 7
Relative Proportion Change of Abundance of Fecal
Microorganisms at Species Level in Cows at different
metabolic states (mean ± standard deviation)¹

| Microorganisms | Norm | MFL | SFL |
|---|---|---|---|
| Lachnospiraceae bacterium | 55.78% | 66.00% | 45.02% |
| Prevotellamultisaccharivorax | 1.38% | 1.07% | 1.90% |
| Bifidobacteriumpseudolongum | 0.73% | 6.30% | 6.04% |

¹Note: Proportion of these microorganisms on the premise that the proportion of the top 20 microorganisms with abundance of the fecal microorganisms in cows is 100% under normal conditions (Norm).

### Example 5: Improvement of Validation Ability by Combined Analysis of Microorganisms-to-Microorganisms

In order to improve the validation ability of microorganisms among groups, the three microorganisms were analyzed in combination. Using binary logistic regression in SPSS, three microbiological variables were arranged and combined in different ways, and the predicted values after combination were fitted, and then ROC analysis was carried out.

TABLE 8
Analysis on Accuracy in Diagnosis of Metabolic
Status (MFLvsNorm) of Cows by different Microorganisms
in Combination at Species Levels

| Microorganisms in Combination | AUC | P value |
|---|---|---|
| Bifidobacteriumpseudolongum + | 0.922 | 0.002 |
| Prevotellamultisaccharivorax + | | |
| Lachnospiraceae bacterium | | |
| Bifidobacteriumpseudolongum + | 0.922 | 0.002 |
| Lachnospiraceae bacterium | | |
| Bifidobacteriumpseudolongum + | 0.889 | 0.004 |
| Prevotellamultisaccharivorax | | |
| Prevotellamultisaccharivorax + | 0.689 | 0.165 |
| Lachnospiraceae bacterium | | |

TABLE 9
Analysis on Accuracy in Diagnosis of Metabolic
Status (SFLvsNorm) of Cows by different Microorganisms
in Combination at Species Levels

| Microorganisms in Combination | AUC | P value |
|---|---|---|
| Bifidobacteriumpseudolongum + | 0.992 | 0.00 |
| Prevotellamultisaccharivorax + | | |
| Lachnospiraceae bacterium | | |
| Bifidobacteriumpseudolongum + | 0.985 | 0.00 |
| Lachnospiraceae bacterium | | |
| Bifidobacteriumpseudolongum + | 0.725 | 0.075 |
| Prevotellamultisaccharivorax | | |
| Prevotellamultisaccharivorax + | 0.767 | 0.035 |
| Lachnospiraceae bacterium | | |

The results showed that the combination of the two microorganisms Bifidobacteriumpseudolongum+Lachnospiraceae bacterium was the best combination in MFLvsNorm group (Table 8). In SFLvsNorm group, the combined diagnosis of the two microorganisms Bifidobacteriumpseudolongum+Lachnospiraceae bacterium was also one of the best diagnostic combinations (Table 9).

In conclusion, Bifidobacteriumpseudolongum, Prevotellamultisaccharivorax and Lachnospiraceae bacterium in cows' feces at species level can be used as non-invasive diagnostic markers for fatty liver in perinatal cows.

In the cow breeding application, the potential fatty liver diseases in cows can be identified and detected by detecting the relative abundance of these three or two of them (Bifidobacteriumpseudolongum+Lachnospiraceae bacterium) in cows' feces. When the abundance of Bifidobacteriumpseudolongum and Lachnospiraceae bacterium in the fecal microorganisms shows an increasing trend at the same time, especially three species (adding Prevotellamultisaccharivorax) are all increasing at the same time, it is necessary to early warn that perinatal cows may be at risk of suffering from fatty liver, and the feeding program should be adjusted or measures should be taken to avoid losses. This biomarker provides a new technology and method for non-invasive detection and diagnosis of fatty liver disease in cows in the future.

### Example 6: Verification of Microorganism Identification Ability

The subjects of this experiment were Chinese Holstein cows within 1-2 weeks after calving. The experiment was conducted in a large-scale dairy farm in Shandong Province. During the experiment, the feeding and management of cows for sample collection were consistent. By preliminary screening by serological test and diagnosis by specific cow liver biopsy, the cows with severe fatty liver (labeled as SFL), cows with moderate fatty liver (labeled as MFL) and cows with normal liver (labeled as Norm) were finally obtained. Liver biopsy diagnosis was based on the percentage of fat deposition cells per unit area after oil red O staining. In severe fatty liver group, moderate fatty liver group and normal liver group, n=12, n=9, and n=10, respectively. Fasting stool samples were collected from three groups of different cows before morning feeding, and frozen in liquid nitrogen for subsequent macroproteomics analysis.

The relative abundance of the top 20 microorganisms at genus level (Table 10) was identified. The abundance change of microorganisms was analyzed by using the ROC analysis in SPSS software based on the relative abundance of microorganisms, and Bifidobacterium, Roseburia and Lachnonaerobacillus at genus level were found in the feces. Their AUC values were 0.833, 0.811 and 0.767, respectively. This showed that the three groups of microorganisms in MFLvsNorm have a high identification ability. (See Table 11) However, AUC of the three groups of microorganisms in SFLvsNorm group is not high.

TABLE 10
Relative Abundance of the top 20 microorganisms at the genus level (mean ± standard deviation)¹

| Microorganism (Genus) | Norm | MFL | SFL |
|---|---|---|---|
| Prevotella | 1.36E+10±6.18E+09 | 1.21E+10±4.48E+09 | 1.05E+10±3.03E+09 |
| Bacteroides | 1.24E+10±3.35E+09 | 1.12E+10±3.62E+09 | 1.04E+10±3.46E+09 |
| Bifidobacterium | 9.95E+08±2.96E+08 | 9.68E+09±1.55E+10 | 4.73E+09±7.58E+09 |
| Treponema | 4.3E+09±2.3E+09 | 4.68E+09±1.15E+09 | 5.55E+09±2.18E+09 |
| Alistipes | 5.81E+09±2.56E+09 | 5.36E+09±1.78E+09 | 4.08E+09±1.63E+09 |
| Romboutsiailealis | 8.46E+08±5.46E+08 | 1.02E+09±3.46E+08 | 8.82E+08±3.49E+08 |
| Turicibactersanguinis | 1.61E+09±9.85E+08 | 1.87E+09±4.88E+08 | 1.29E+09±6.17E+08 |
| Cellulosilyticumlentocellum | 1.11E+09±1.02E+09 | 8.37E+08±4.72E+08 | 5.09E+08±4.42E+08 |
| Alloprevotella | 7.07E+08±3.07E+08 | 5.31E+08±2.22E+08 | 5.59E+09±4.18E+08 |
| Roseburia | 4.48E+08±1.5E+08 | 6.33E+08±1.66E+08 | 5.41E+08±3.91E+08 |
| Olsenella | 3.88E+08±1.78E+08 | 3.8E+08±1.99E+08 | 2.38E+08±3.18E+08 |
| Paraprevotella | 4.68E+08±3.37E+08 | 4.37E+08±2.41E+08 | 5.18E+08±4.13E+08 |
| Clostridium | 6.05E+08±2.8E+08 | 6.28E+08±1.95E+08 | 3.62E+08±1.72E+08 |
| Butyrivibrio | 4.09E+08±1.87E+08 | 6.91E+08±4.32E+08 | 2.16E+08±9.6E+07 |
| Bradyrhizobium | 1.96E+08±2.48E+08 | 3.72E+08±2.91E+08 | 4.3E+07±1.42E+08 |
| Dorea | 2.86E+08±1.72E+08 | 7.01E+08±7.09E+08 | 1.72E+08±1.15E+08 |
| Terrisporobacter | 3.03E+08±5.8E+08 | 1.86E+08±3.24E+08 | 1.04E+08±6.4E+07 |
| Phascolarctobacteriumfaecium | 2.67E+08±2.96E+08 | 3.14E+08±1.9E+08 | 1.91E+08±1.47E+08 |
| Lachnoanaerobaculum | 2.72E+08±9.3E+07 | 3.71E+08±1.26E+08 | 2.13E+08±1.0E+08 |
| Ruminococcus | 2.03E+08±9.9E+07 | 2.64E+08±8.8E+07 | 2.88E+08±1.17E+08 |

¹Note:
Top 20 Microorganisms with Abundance at Genus Level of Fecal Microorganisms in Cows

TABLE 11
Validation of Accuracy of Fecal Microorganisms at Genus Level
In Diagnosing the Metabolic Status of Cows (MFLvsNorm)¹

| Microorganism | | | |
|---|---|---|---|
| (Genus) | AUC | AUC P value | MFL/Norm P value |
| Bifidobacterium | 0.833 | 0.014 | 0.0755 |
| Roseburia | 0.811 | 0.022 | 0.0144 |
| Lachnoanaerobaculum | 0.767 | 0.05 | 0.0447 |
| Treponema | 0.7 | 0.142 | 0.3395 |
| Dorea | 0.7 | 0.142 | 0.0709 |
| Turicibactersanguinis | 0.689 | 0.165 | 0.2502 |
| Butyrivibrio | 0.678 | 0.191 | 0.0578 |
| Bradyrhizobium | 0.667 | 0.221 | 0.1008 |
| Ruminococcus | 0.656 | 0.253 | 0.0991 |
| Phascolarcto | 0.644 | 0.288 | 0.3475 |
| Bacteriumfaecium | | | |
| Romboutsiailealis | 0.622 | 0.369 | 0.2225 |
| Clostridium | 0.544 | 0.744 | 0.4246 |
| Paraprevotella | 0.5 | 1 | 0.4134 |
| Alistipes | 0.489 | 0.935 | 0.3385 |
| Prevotella | 0.467 | 0.806 | 0.2850 |
| Cellulosilyticum | 0.456 | 0.744 | 0.2391 |
| Lentocellum | | | |
| Olsenella | 0.444 | 0.683 | 0.4684 |
| Bacteroides | 0.433 | 0.624 | 0.2475 |

¹Note:
Top 20 Microorganisms with Abundance at Genus Level of Fecal Microorganisms in Cows

TABLE 12
Validation of Accuracy of Fecal Microorganisms at Genus Level
In Diagnosing the Metabolic Status of Cows (SFLvsNorm)¹

| | | | SFL/Norm P |
|---|---|---|---|
| microorganism | AUC | AUC P value | value |
| Ruminococcus | 0.717 | 0.086 | 0.046 |
| Treponema | 0.658 | 0.21 | 0.121 |
| Bifidobacterium | 0.633 | 0.291 | 0.065 |
| Romboutsiailealis | 0.592 | 0.468 | 0.433 |
| Roseburia | 0.55 | 0.692 | 0.238 |
| Paraprevotella | 0.492 | 0.947 | 0.385 |
| Terrisporobacter | 0.483 | 0.895 | 0.164 |
| Phascolarctobacteriumfaecium | 0.475 | 0.843 | 0.247 |
| Turicibactersanguinis | 0.458 | 0.742 | 0.206 |
| Prevotella | 0.342 | 0.21 | 0.097 |
| Bradyrhizobium | 0.338 | 0.199 | 0.061 |
| Alistipes | 0.308 | 0.129 | 0.050 |
| Lachnoanaerobaculum | 0.3 | 0.114 | 0.094 |
| Dorea | 0.292 | 0.099 | 0.053 |
| Bacteroides | 0.283 | 0.086 | 0.104 |
| Clostridium | 0.283 | 0.086 | 0.019 |
| Cellulosilyticumlentocellum | 0.267 | 0.065 | 0.062 |
| Alloprevotella | 0.267 | 0.065 | 0.208 |
| Butyrivibrio | 0.217 | 0.025 | 0.007 |
| Olsenella | 0.133 | 0.004 | 0.100 |

¹Note:
Top 20 Microorganisms with Abundance at Genus Level of Fecal Microorganisms in Cows

### Example 7: Abundance Change of Microorganisms in Three Groups of Norm-MFL-SFL

The three groups of microorganisms Bifidobacterium, Roseburia, and Lachnonanerobaculum screened by ROC showed that the abundance of Bifidobacterium in the diseased group increased significantly from 2.20% in Norm to 18.51% in MFL to 11.42% in SFL. The abundance changes of Roseburia and Lachnonanaerobaculum in Norm-MFL-SFL also increased in the diseased group, but the abundance accounted for a small proportion (Table 13).

TABLE 13
Relative Proportion Change of Abundance of Fecal
Microorganisms at Genus Level in Cows in different
metabolic states (mean ± standard deviation)¹

| Microorganisms | Norm | MFL | SFL |
|---|---|---|---|
| Bifidobacterium | 2.20% | 18.51% | 11.42% |
| Roseburia | 0.99% | 1.21% | 1.31% |
| Lachnoanaerobaculum | 0.60% | 0.71% | 0.51% |

¹Note:
Proportion of these microorganisms on the premise that the proportion of the top 20 microorganisms with abundance of the fecal microorganisms in cows is 100% under normal conditions (Norm).

### Example 8: Improvement of Validation Ability by Combination of Microbiological Markers

In order to improve the validation ability of microorganisms among groups, the three microorganisms at genus level were analyzed in combination. Using binary logistic regression in SPSS, three microbiological variables were arranged and combined in different ways, and the predicted values after combination were fitted, and then ROC analysis was carried out. Finally, it is concluded that the combined performance of Lachnonanerobaculum, Roseburia and Bifidobacterium is the best at genus level. In MFLvsNorm group, the AUC of three groups of microorganisms analyzed in combination reached 1, and in SFLvsNorm group, the AUC of the two groups of Lachnonaerobaculum and Bifidobacterium analyzed in combination reached 0.867.

TABLE 14
Analysis on Accuracy in Diagnosis of Metabolic
Status (MFLvsNorm) of Cows by different Microorganisms
in Combination at Genus Levels

| Combined category of microorganisms | AUC | P value |
|---|---|---|
| Lachnoanaerobaculum + Roseburia + Bifidobacterium | 1 | 0 |
| Roseburia + Bifidobacterium | 0.967 | 0.001 |
| Lachnoanaerobaculum + Bifidobacterium | 0.944 | 0.001 |
| Lachnoanaerobaculum + Roseburia | 0.833 | 0.014 |

TABLE 15
Analysis on Accuracy in Diagnosis of Metabolic
Status (SFLvsNorm) of Cows by different Microorganisms
in Combination at Genus Levels

| Combined category of microorganisms | AUC | P value |
|---|---|---|
| Lachnoanaerobaculum + Roseburia + Bifidobacterium | 0.867 | 0.004 |
| Lachnoanaerobaculum + Bifidobacterium | 0.867 | 0.004 |
| Roseburia + Bifidobacterium | 0.792 | 0.021 |
| Lachnoanaerobaculum + Roseburia | 0.742 | 0.056 |

Table 11 The data obtained from ROC analysis of single microorganism is the result of MFLvsNorm group. Three groups of microorganisms, namely Lachnoanaerobaculum, Roseburia, and Bifidobacterium have good effects in MFLvsNorm, but the scores of these three microorganisms in SFLvsNorm group are not high (see Table 12). However, the AUC of the three groups of microorganisms analyzed in combination in SFLvsNorm group is higher than the AUC value of each microorganism.

In the cow breeding application, the potential fatty liver diseases in cows can be identified and detected by detecting the relative abundance of these three microorganisms in cows' feces. When the abundances of Bifidobacterium and Lachnonanerobaculum in fecal microorganisms show an increasing trend at the same time, especially when the abundances of of the three microorganisms (adding Roseburia) are increasing at the same time, it is necessary to early warn the possibility of metabolic disorders, and it is necessary to adjust the feeding program or take adjustment measures in time to effectively avoid losses. This biomarker provides a new technology and method for non-invasive detection and diagnosis of fatty liver disease in cows in the future.

The above descriptions are only the preferred examples of the present disclosure, and is not intended to limit thereto. For those skilled in the art, various modifications and changes can be made to the present disclosure. Any modifications, equivalent substitutions, improvements, and the like made within the spirit and principle of the present disclosure shall be included into the protection scope of the present disclosure.

What is claimed is:

1. A microorganism of at least one species of the following 1)-6) as a microbiological marker for preparation of a reagent or a kit for diagnosing the fatty liver diseases in perinatal cows:
1) Bifidobacteriumpseudolongum;
2) Prevotellamultisaccharivorax;
3) Lachnospiraceae bacterium;
4) Lachnoanaerobaculum;
5) Roseburia; and
6) Bifidobacterium.

2. The microbiological marker according to claim 1, wherein the microbiological marker is a combination of the three Bifidobacteriumpseudolongum, Prevotellamultisaccharivorax and Lachnospiraceae bacterium;
or, the microbiological marker is a combination of the two Bifidobacteriumpseudolongum and Lachnospiraceae bacterium;
or, the microbiological marker is a combination of at least two of Lachnoanaerobaculum, Roseburia, and Bifidobacterium;
or, the microbiological marker is a combination of Lachnoanaerobaculum, Roseburia, and Bifidobacterium.

3. A reagent for detecting microbiological markers in cows' metabolites for preparation of products for non-invasively identifying fatty liver diseases in perinatal cows;
the microbiological markers being a combination of one or more of Bifidobacteriumpseudolongum, Prevotellamultisaccharivorax, Lachnospiraceae bacterium, Lachnoanaerobaculum, Roseburia and Bifidobacterium.

4. The reagent according to claim 3, wherein the microbiological marker is a combination of the three Bifidobacteriumpseudolongum, Prevotellamultisaccharivorax and Lachnospiraceae bacterium;
or, the microbiological marker is a combination of the two Bifidobacteriumpseudolongum and Lachnospiraceae bacterium;
or, the microbiological marker is a combination of at least two of Lachnoanaerobaculum, Roseburia, and Bifidobacterium;
or, the microbiological marker is a combination of Lachnoanaerobaculum, Roseburia, and Bifidobacterium.

5. The reagent according to claim 3, wherein the reagent is one for detecting the relative abundance of the microbiological markers in cows' metabolites.

6. The reagent according to claim 3, wherein the reagent is one for Metagenomics sequencing, 16S sequencing, qPCR sequencing or Metaproteomics sequencing.

7. The reagent according to claim 5, wherein the reagent is one for Metagenomics sequencing, 16S sequencing, qPCR sequencing or Metaproteomics sequencing.

8. The reagent according to claim 3, wherein the cows' metabolite is feces.

9. The reagent according to claim 4, wherein the cows' metabolite is feces.

10. The reagent according to claim 3, wherein the method for non-invasively identifying fatty liver diseases in perinatal cows comprises:
(1) collecting feces from perinatal cows to be tested;
(2) detecting the relative abundance of microbiological markers in cows' feces; and
(3) diagnosing/identifying whether the cows to be tested suffer from fatty liver diseases based on the relative abundance of the detected microbiological markers or identifying the fatty liver diseases of the perinatal cows to be tested based on the relative abundance of the detected microbiological markers for diagnosis or early warning.

11. The reagent according to claim 10, wherein the feces are the feces before feeding in the morning.

12. The reagent according to claim 4, wherein the method for non-invasively identifying fatty liver diseases in perinatal cows comprises:
(1) collecting feces from perinatal cows to be tested;
(2) detecting the relative abundance of microbiological markers in cows' feces; and
(3) diagnosing/identifying whether the cows to be tested suffer from fatty liver diseases based on the relative abundance of the detected microbiological markers or identifying the fatty liver diseases of the perinatal cows to be tested based on the relative abundance of the detected microbiological markers for diagnosis or early warning.

13. The reagent according to claim 12, wherein the feces are the feces before feeding in the morning.

14. A method for diagnosing fatty liver diseases in perinatal cows, wherein it comprises the following steps:
(1) collecting feces from perinatal cows to be tested;
(2) detecting the relative abundance of microbiological markers in cows' feces; and
(3) diagnosing/identifying whether the cows to be tested suffer from fatty liver diseases based on the relative abundance of the detected microbiological markers or identifying the fatty liver diseases in the perinatal cows to be tested based on the relative abundance of the detected microbiological markers for diagnosis or early warning;
the microbiological markers being at least one of Bifidobacteriumpseudolongum, Prevotellamultisaccharivorax, Lachnospiraceae bacterium, Lachnoanaerobaculum, Roseburia or Bifidobacterium.

15. The method according to claim 14, wherein the microbiological marker is a combination of the three Bifidobacteriumpseudolongum, Prevotellamultisaccharivorax and Lachnospiraceae bacterium;
or, the microbiological marker is a combination of the two Bifidobacteriumpseudolongum and Lachnospiraceae bacterium;
or, the microbiological marker is a combination of at least two of Lachnoanaerobaculum, Roseburia, and Bifidobacterium;
or, the microbiological marker is a combination of Lachnoanaerobaculum, Roseburia, and Bifidobacterium.

16. The method according to claim 14, wherein the relative abundance of the microbiological markers in cows' feces is detected by Metagenomics sequencing, 16S sequencing, qPCR sequencing or Metaproteomics sequencing.
